(19) United States
(12) Patent Application Publication
(10) Pub. No.: US 2017/0035847 A1
(43) Pub. Date: Feb. 9, 2017

(54) COMPOSITIONS AND METHODS FOR TREATING INFLAMMATION-ASSOCIATED DISORDERS OF THE GASTROINTESTINAL TRACT

(71) Applicant: TherapyX, Inc., Buffalo, NY (US)

(72) Inventors: Nejat K. Egilmez, Louisville, KY (US); Edith Mathiowitz, Brookline, MA (US); Dominik L. Auci, Louisville, KY (US)

(21) Appl. No.: 15/230,866

(22) Filed: Aug. 8, 2016

Related U.S. Application Data
(60) Provisional application No. 62/201,733, filed on Aug. 6, 2015.

(51) Int. Cl. A61K 38/18; A61K 9/50; A61K 31/203

(52) U.S. Cl. CPC A61K 38/1841; A61K 31/203; A61K 9/50

(57) ABSTRACT

Compositions and methods for treating inflammatory disorders of the GI tract are provided. The compositions include a therapeutically effective amount of TGFβ and ATRA (all-trans retinoic acid), in a pharmaceutically acceptable carrier suitable for oral administration. TGFβ and ATRA are preferably encapsulated in microspheres.

The compositions are useful in alleviating symptoms in subject with an inflammatory disease of the gastrointestinal tract. Exemplary subjects include subjects with Chrohns disease, ulcerative colitis, gastritis, irritable bowel syndrome, ileitis, etc.

The composition is administered to the subject following a therapeutically effective regimen, for length of time resulting in an improvement in one or more symptoms associated with inflammatory disorders of the GI tract.

### CROSS REFERENCE TO RELATED APPLICATIONS

This application claims benefit of U.S. Provisional Application No. 62/201,733, filed December Aug. 6, 2015, which is hereby incorporated herein by reference in its entirety.

### STATEMENT REGARDING FEDERALLY SPONSORED RESEARCH

This invention was made with government support under 5R44AI080009-04 awarded by National Institute of Health. The government has certain rights in the invention

### FIELD OF THE INVENTION

The invention is generally directed to compositions and methods for treating disorders of the gastrointestinal tract associated with inflammation.

### BACKGROUND OF THE INVENTION

Inflammatory bowel disease (IBD) is a chronic disorder of the GI tract causing significant morbidity for over 1.4 million Americans. IBD is a broad term that describes conditions with chronic or recurring immune response and inflammation of the gastrointestinal (GI) tract. Symptoms include diarrhea, nausea, abdominal pain, weight loss, increased risk for colorectal cancer, and can be fatal. The two most common inflammatory bowel diseases are ulcerative colitis and Crohn's disease. Inflammation can affect the entire digestive tract in Crohn's disease and primarily only the large intestine and rectum in ulcerative colitis.

Although etiologies of IBD are incompletely understood, genetic, immunologic and environmental factors all make significant contributions. Human and animal studies implicate abnormal responses to commensal microflora and perturbed local immune homeostasis. Cytokines have a crucial role in the pathogenesis of inflammatory bowel diseases (IBDs), such as Crohn's disease and ulcerative colitis, where they control multiple aspects of the inflammatory response. In particular, the imbalance between pro-inflammatory and anti-inflammatory cytokines that occurs in IBD impedes the resolution of inflammation and instead leads to disease perpetuation and tissue destruction. In CD, activated Type 1 T helper (Th) cells have been implicated, although emerging evidence suggests important roles for IL-17-producing CD4+ T (Th17) cells. In CD, for example, there is increased production in the intestinal mucosa of the Th17 cytokine interleukin-17 and the Th cytokines interferon-γ and TNF-α. In UC, on the other hand, interleukin(IL)-1, IL-5, IL-6, IL-8 and TNF-α responses are increased, but not IL-4 or interferon γ, suggesting abnormal macrophage and monocyte activity (Abraham, et al., New England Journal of Medicine, 361(21):2066-2078 (2009); Podolsky, et al., New England Journal of Medicine, 347(6):417-429 (2002); Yen, et al., J. Clin. Invest., 116(5):1310-1316 (2006); Cho, et al., Nat. Rev. Immunol., 8(6):458-466 (2008); Hue, et al., J. Exp Med., 203(11):2473-2483 (2006); Maynard, et al., Immunity, 31(3):339-400 (2009)).

Conventional therapies for IBD, including CD and UC fall into three groups: salicylates, immunosuppressants and antibiotics. Although these are effective in treating active disease, remissions are often short-lived and associated with significant side effects. Despite therapy, 80% and 45% of CD and UC patients (respectively) require surgery (Abraham, et al., New England Journal of Medicine, 361(21):2066-2078 (2009); Podolsky, et al., New England Journal of Medicine, 347(6):417-429 (2002); Kraus, et al., Current opinion in pharmacology, 9(4):405-410 (2009); Grivennikov, et al., Sem. Immunol., 35(2):229-244 (2013); Risques, et al., Cancer research, 71(5):1669-1679 (2011); Podolsky, et al., Best practice & research Clinical gastroenterology, 16(6):933-943 (2002).

A therapeutic group of anti-inflammatory agents has emerged, the so-called biological response modifiers or ‘biologics’, macromolecules that target inflammatory lymphocytes or the cytokines they produce (Rutgeerts, et al., Gastroenterology, 136(4):1182-1197 (2009)). One such molecule, infliximab, a chimeric anti-human tumor necrosis factor (TNF)α antibody, was granted FDA approval in 1998 with a high response rate, significant mucosal and fistula healing and long-term remissions in Crohn's disease. Other emerging biologics include anti-p40, anti-p19, anti-IL-12, anti-IL-17 and anti-alpha 4 integrin antibodies (Rutgeerts, et al., Gastroenterology, 136(4):1182-1197 (2009); Neurath, et al., Immunity, 31(3):357-361 (2009)). However, an estimated 30% of patients will not respond to biologics and of those who initially respond, 50% relapse within a year. Biologics have had only modest impact on surgical intervention rates (Cannom, et al., The American Surgeon, 75(10):976-980 (2009)). Thus, the need for a deeper understanding of pathogenesis and novel, targeted therapies remains acute.

Regulatory T cells (Ts) and mucosal dendritic cells (DC) help maintain intestinal immune homeostasis and represent attractive therapeutic targets (Maynard, et al., Immunity, 31(3):339-400 (2009); Barnes, et al., Immunity, 31(3)401-411 (2009); Izcue, et al., Immunological Reviews, 212:256-271 (2006); Coombes, et al., J. Exp. Med., 204(8):1757-1764 (2007); Sun, et al., J. Exp. Med., 204(8): 1775-1785 (2007)). T_{regs} are a subpopulation of T cells including CD4+ CD25+ forkhead box P3 (Foxp3)+ T cells, Tr1, Th3, and CD8+ T_{regs}. They serve to suppress the immune system and maintain self-tolerance. Adoptively transferred CD4+ CD25+ Foxp3+ transforming growth factor (TGF)β-dependent T_{regs} prophylactically and therapeutically limit IBD via direct suppression of T-effector priming activity and innate effector function (Coombes, et al., J. Exp. Med., 204(8): 1757-1764 (2007); Liu, et al., J. Immunol., 171(10):5012-5017 (2003); Mottet, et al., J. Immunol., 170(8):3939-3943 (2003)). Separately, mucosal DCs produce retinoic acid, an essential cofactor in TGFβ-dependent T_{regs} generation in the gut (Coombes, et al., J. Exp. Med., 204(8):1757-1764 (2007); Sun, et al., J. Exp. Med., 204(8):1775-1785 (2007); Sun, et al., J. Exp. Med., 204(8):1775-1785 (2007)). This retinoic acid requirement was linked to induction of Foxp3 expression in T-cells (Elias, et al., Blood, 111(3): 1013-1020 (2008)). Thus, these molecules represent potential therapeutic agents to treat IBD by activation and expansion of gut T_{regs}. However, TGFβ therapy presents with concerns related to fibrosis, the development of which is a serious complication in IBD. TGF-β1 is considered the most potent mediator of fibrosis through its direct effect on fibroblasts (Reider, et al., Curr. Opin Gastroenterol., 24:462-468 (2008)). Consistent with the known activities of TGF-β1, chronic administration (or transgenic overexpression) in animal models has led to side effects such as interstitial and hepatic fibrosis (Prud'homme, et al., Lab. Invest., 87(11):1077-1091 (2007)).

There is still a need for compositions for treating inflammatory disorders of the GI, while minimizing undesirable side effects.

It is an object of the present invention to provide compositions for treating inflammatory disorders of the GI tract.

It is also an object of the present invention to provide a method of treating inflammatory disorders of the GI tract.

### SUMMARY OF THE INVENTION

Oral compositions for reducing one or more symptoms associated with inflammatory disorders of the GI tract are provided. The compositions include a therapeutically effective amount of TGFβ and ATRA (all-trans retinoic acid), in a pharmaceutically acceptable carrier suitable for oral administration. TGFβ and ATRA are preferably encapsulated in microspheres. More preferably, TGFβ and ATRA are encapsulated separately and then combined in a suitable carrier. In a most preferred embodiment, ATRA is encapsulated into poly(lactic-co-glycolic)(PLGA) acid microspheres, and TGFβ is encapsulated into poly(lactic acid) (PLA) microspheres using phase inversion nano-encapsulation (PIN®.

Also disclosed is a method of treating a subject in need thereof, with compositions containing a therapeutically effective amount of TGFβ and ATRA. The composition is administered to the subject following a therapeutically effective regimen, for a length of time resulting in an improvement in one or more symptoms associated with inflammatory disorders of the GI tract. For example, the composition can be administered one time per week, two times per week, or three times per week. In a preferred embodiment, the composition is administered three times per week. The subject is treated for a length of time effective to ameliorate symptoms.

The compositions are useful in alleviating symptoms in subject with an inflammatory disease of the gastrointestinal tract.

### BRIEF DESCRIPTION OF THE DRAWINGS

FIGS. 1A and 1B show in vitro release profile and bioactivity of TGFβ over 4 days. FIG. 1A shows TGFβ1 release. FIG. 1B shows TGFβ bioactivity for the 15 minute release samples from FIG. 1A, expressed as the equivalent of unencapsulated recombinant TGFβ concentration. Data are averages of 5 different batches. Error bars=SD.

FIG. 2 shows the effect of storage time and temperature on TGFβ biological activity. Data are averages of triplicates from one of at least three experiments with similar results expressed as % activity of the unstored sample. There were no statistically-significant differences between any of the time-points. Error bars=SE.

FIGS. 3A-3D show the therapeutic activity of oral TGFβ or ATRA loaded particles given alone versus together in the SCID mouse adoptive CD4+ CD25− T-cell transfer model of IBD measured as % change in body weight relative to day of first treatment (FIG. 3A); effect on colon length/weight (FIG. 3B); effect on serum amyloid A (SAA) μg/mL as determined by ELISA (FIG. 3C); or histological score (FIG. 3D). Error bars=SE, n=4 per group. Significance: *, **, ***, **** denote p<0.05, 0.01, 0.001 and 0.0001, respectively.

FIGS. 4A-4C show dose response of TGFβ in the SCID mouse adoptive CD4+ CD25− T-cell transfer model of IBD, measured as: % change in body weight relative to day of first treatment (FIG. 4A); colon length/weight (FIG. 4B); or serum amyloid A (SAA) μg/mL as determined by ELISA (FIG. 4C). Error bars=SE, n=4-5 per group. Significance: *, **, *** and **** denote p<0.05, 0.01, 0.001 and 0.0001, respectively.

FIGS. 5A-5C show dose response of ATRA in the SCID mouse adoptive CD4+ CD25− T-cell transfer model of IBD, measured as: % change in body weight relative to day of first treatment (FIG. 5A); colon length/weight (FIG. 5B); or serum amyloid A (SAA) μg/mL as determined by ELISA (FIG. 5C). Error bars=SE, n=5 per group. Significance: *, **, *** and **** denote p<0.05, 0.01, 0.001 and 0.0001, respectively.

FIGS. 6A-6C show the effect of Dose Schedule on TGFβ/ATRA Therapeutic Activity in the SCID mouse adoptive CD4+ CD25− T-cell transfer model of IBD, expressed as: % change in body weight relative to day of first treatment (FIG. 6A); colon length/weight (FIG. 6B); or serum amyloid A (SAA) μg/mL as determined by ELISA (FIG. 6C). Error bars=SE, n=4 per group. Significance: *, **, *** and **** denote p<0.05, 0.01, 0.001 and 0.0001, respectively.

FIGS. 7A-7E show the therapeutic activity of TGFβ/ATRA loaded particles in the SCID mouse adoptive CD4+ CD25− T-cell transfer model of IBD, expressed as: average disease score (FIG. 7A); % change in body weight relative to day of first treatment (FIG. 7B); serum amyloid A (SAA) μg/mL as determined by ELISA (FIG. 7C); colon weight to length ratio (FIG. 7D); or histological score with representative colon histology for each group shown (FIG. 7E). Micrographs=100× magnification. FIG. 7A. Differences between the treated and untreated groups on days 32-43 were significant (p<0.049). FIG. 7B. Differences between groups were significant between days 5-18 (p<0.039). Error bars=SE. Significance: *, **, *** and **** denote p<0.05, 0.01, 0.001 and 0.0001, respectively.

FIGS. 8A and 8B show efficacy and toxicity of long term TGFβ/ATRA microsphere therapy. FIG. 8A shows colon weight-to-length ratio. Values from age-matched naive controls are shown for comparison. Error bars=standard error [SE], n=5, 6, 6, 7 for naive, untreated [ACT], 2-week treatment, and 8-week treatment groups, respectively. Significance: * denotes p<0.05. FIG. 8B shows the 8-week survival for control [ACT only] and 8-week TGFβ/ATRA groups. The difference between the groups was significant [p=0.024] by log-rank analysis [n=11 and 7 for control and TGFβ/ATRA groups, respectively]. FIG. 8C is a bar graph showing the effect of oral treatment with TGFβNanoCap on levels of TGFβ in the colon.

FIG. 9 shows the effect of TGFβ/ATRA treatment on CD4+ CD25+ Foxp3+ expression by lamina propria T cells in the SCID mouse adoptive CD4+ CD25− T-cell transfer model of IBD. The average MFI per group is shown in the bar graph. Error bars=SE, n=5 per group. Significance: p=0.0015.

FIGS. 10A-10E show the therapeutic activity of TGFβ/ATRA-loaded particles in the murine model of dextran sodium sulphate-[DSS]-induced colitis. Data are expressed as: average cumulative disease score (FIG. 10A); % change in body weight relative to day of first treatment (FIG. 10B); serum amyloid A [SAA] μg/ml as determined by ELISA (FIG. 10C); colon weight-to-length ratio (FIG. 10D); and histological score (FIG. 10E). Error bars=standard error [SE]. Significance: *, **, ***, and **** denote p<0.05, 0.01, 0.001, and 0.0001, respectively.

### DETAILED DESCRIPTION OF THE INVENTION

Acid-induced hydrolysis and enzymatic degradation often prevent effective oral delivery of bioactive macromolecules, such as TGFβ (Goldberg, et al., Nat. Rev. Drug. Dis., 2(4):289-295 (2003); Langer, et al., Nature, 428(6982):487-492 (2004)). Encapsulation of labile biologics into biodegradable polymer based microspheres provides protection from stomach acids and proteases, allowing safe passage into the lumen of the GI tract for uptake (Egilmez, et al., Endocrine, Metabolic & Immune Disorders Drug Targets, 7(4):266-270 (2007); Mathiowitz, et al., Nature, 386(6623):410-414 (1997)).

The development of a formulation which can deliver TGFβ orally and which can provide targeted local delivery of low doses of TGFβ to the GI tract can significantly improve the efficacy of TGFβ treatment in IBD patients while avoiding the potential problems associated with systemic TGFβ administration and gene therapy approaches. Formulations with these properties are disclosed herein.

### I. DEFINITIONS

The term “controlled release” or “modified release” refers to a release profile in which the active agent release characteristics of time course and/or location are chosen to accomplish therapeutic or convenience objectives not offered by conventional dosage forms such as solutions, suspensions, or promptly dissolving dosage forms. Delayed release, extended release, and pulsatile release and their combinations are examples of modified release.

“Pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Therapeutically effective” or “effective amount” as used herein means that the amount of the composition used is of sufficient quantity to ameliorate one or more causes or symptoms of a disease or disorder. Such amelioration only requires a reduction or alteration, not necessarily elimination. As used herein, the terms “therapeutically effective amount” and “pharmaceutically effective amount” are synonymous. One of skill in the art can readily determine the proper therapeutically effective amount.

A “subject” or “patient” refers to a human, primate, non-human primate, laboratory animal, farm animal, livestock, or a domestic pet.

The term “treat” or “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

### II. COMPOSITIONS

The compositions include a therapeutically effective amount of TGFβ and ATRA, in a pharmaceutically acceptable carrier suitable for oral administration. TGFβ and ATRA are preferably encapsulated in nano or microspheres. More preferably, TGFβ and ATRA encapsulated separately and then combined in a suitable carrier. In a most preferred embodiment, ATRA is encapsulated into poly(lactic-co-glycolic)(PLGA) acid microspheres, and TGFβ is encapsulated into poly(lactic acid) (PLA) microspheres phase inversion nano-encapsulation (PIN®).

The compositions reduce one or more symptoms associated with inflammatory disorders of the GI tract when compared to TGFβ or ATRA administered alone using the same route and at the same concentrations. For example, the composition is effective to reduce disease associated weight loss, disease associated elevation of SAA levels and/or disease associated fibrosis when compared to TGFβ or ATRA administered alone. In a preferred embodiment, the composition is effective to reduce disease associated weight loss, disease associated elevation of SAA levels and disease associated fibrosis when, when compared to TGFβ or ATRA administered alone using the same route of administration. For example, the compositions are effective to reduce disease associated intestinal fibrosis, in the absence therapy associated/induced fibrosis. In a particularly preferred embodiment, the composition is effective to reduce endogenous TGFβ levels in the colon when compared to a control subject not administered the composition. In some particularly preferred embodiments, the oral doses of TGFβ or ATRA do not exceed an oral dose equivalent to 60 mg/kg or 30 mg/kg in rats, respectively.

A. TGFβ

Transforming growth factor β (TGFβ) is a highly pleiotropic cytokine that in mammals exists in three isoforms (TGFβ1, TGFβ2 and TGFβ3). Practically all cells have receptors for the TGFβs, and at least one of the isoforms is produced in all tissues. TGFβ exerts broad anti-inflammatory and immunosuppressive effects. TGFβ is an important differentiation factor (along with IL-2) for some regulatory T cells (denoted Tr or Treg cells) that exert powerful and diverse immunosuppressive effects. The compositions disclosed herein can include TGFβ, TGFβ2 and/or TGFβ3. In a most preferred embodiment the compositions include TGFβ1. TGFβ is present in an amount, which, in combination with ATRA, is effective to reduce one or more symptoms associated with inflammatory disorders of the gastrointestinal tract, for example, the severity of inflammation, extent of inflammation, disease associated fibrosis (determined for example by colon weight to length ratio), disease associated weight loss, serum amyloid A levels etc. An effective amount of TGFβ1 for example, is in a range of 1-20 μg, preferably in a range of 2-10 μg in mice, a dosage required to provide an active pharmaceutical ingredient (API) dosage range of 0.04 mg/kg-0.8 mg/kg, in mice. One can readily determine the equivalent effective dose in other species, for example, domestic animals and humans, using techniques that are available in the art (reviewed in Sharma, et al., Br J Pharmacol., 157(6): 907-921(2009)).

B. ATRA

In contrast to TGFβ, ATRA is a small molecule with significant oral bioavailability and wider storage options (Saadeddin, et al., The AAPS J., 6(1):1-9 (2004)). Nanoparticulate ATRA has been used to suppress Th17 cells and induce regulatory T cells in vitro (Capurso, et al., Self Nonself., 1(4):335-340 (2010)). In addition, various studies report the efficacy of ATRA alone in IBD models following intraperitoneal (IP) administration. For example, Collins, et al., (Gastroenterology, 141(5):1821-1831 (2011)) administered twice weekly ATRA injections (300 μg given IP) in mice that overproduce tumor necrosis factor (TNF) and develop chronic ileitis (TNFΔARE mice). Bai, et al., (J. Interferon and Cytokine Res., 30(6):399-406 (2010)) administered ATRA by IP in a trinitrobenzene sulphonic acid (TNBS) model of colitis. Hong, et al., Immunol Lett, 162(1 Pt A):34-40 (2014)) administered ATRA by IP in the mouse model of DSS induced colitis.

Systemically administered ATRA is also used to treat severe cystic acne (also known as nodular acne) that has not responded to other treatment (Prevost, et al., J. Pediatric. Adol. Gynecol., 26(5):290-293 (2013)) as well as acute promyelocytic leukemia APML (Fenaux, et al., Blood, 82(11):3241-3249 (1993); Tallman, et al., New. Eng. J. Med., 337(15): 1021-1028 (1997); Freemantle, et al., Oncogene, 22(47):7305-7315 (2003). Despite its effectiveness, it has been associated with depression, suicidality and teratogenicity (Prevost, et al., J. Pediatric. Adol. Gynecol., 26(5):290-293 (2013)). Alternate approaches avoiding systemic administration of ATRA have employed immune-targeting of liposomal retinoids (Ozpolat, et al., Leukemia & lymphoma, 43(5):933-941 (2002)) or retinoid aerosolization (Wang, et al., Clin. Cancer Res., 6(9):3636-3645 (2000)). Choi, et al., (Int. J. Pharmaceutics, 320(1-2):45-52 (2006) administered 50 mg/kg ATRA micro-particles via a subcutaneous injection within the context of epithelial carcinogenesis.

The compositions disclosed herein include amounts of ATRA which are effective in an oral combination therapy using ATRA and TGFβ, to reduce one or more symptoms associated with inflammatory disorders of the GI tract. An effective amount of ATRA for use in an oral combination therapy with TGFβ, is in a range of 1-100 μg, preferably in a range of 5-40 μg, and even more preferably, between 5 and 20 μg in mice. The compositions disclosed herein provide an active pharmaceutical ingredient (API) dosage range of 0.04 mg/kg-4 mg/kg, in mice. One can readily determine the equivalent effective dose in other species using techniques that are available in the art (reviewed in Sharma, et al., Br J Pharmacol., 157(6): 907-921(2009)).

C. Enteral Formulations

The compounds described herein are formulated for enteral administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients.

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof. Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (POLYPLASDONE® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

(a) Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation in to the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended release coatings. The coating or coatings may also contain the compounds and/or additional active agents.

(1) Extended Release Dosage Forms

The extended release formulations are generally prepared as diffusion or osmotic systems, which are known in the art. A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and CARBOPOL® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid)(anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the trade name EUDRAGIT®. In further preferred embodiments, the acrylic polymer comprises a mixture of two acrylic resin lacquers commercially available from Rohm Pharma under the trade names EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral (meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multiparticulate systems formed to include the same are swellable and permeable in aqueous solutions and digestive fluids.

The polymers described above such as EUDRAGIT® RL/RS may be mixed together in any desired ratio in order to ultimately obtain a sustained-release formulation having a desirable dissolution profile. Desirable sustained-release multiparticulate systems may be obtained, for instance, from 100% EUDRAGIT® RL, 50% EUDRAGIT® RL and 50% EUDRAGIT® RS, and 10% EUDRAGIT® RL and 90% EUDRAGIT® RS. One skilled in the art will recognize that other acrylic polymers may also be used, such as, for example, EUDRAGIT® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

(2) Delayed Release Dosage Forms

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the trade name EUDRAGIT® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### III. METHODS OF MAKING AND USING

A. Methods of Making

Encapsulation of labile biologics into biodegradable polymers provides protection from the acidic environment and the proteases of the stomach and allows safe passage into the lumen of the GI tract for uptake (Yamanaka, et al., J. Biomater. Sci. Polym Ed, 19:1549-70 (2008). Until recently, this technology primarily targeted small molecule drugs and peptides because larger bioactive macromolecules are generally more highly sensitive to organic solvents and mechanical agitation techniques that are used during encapsulation. PIN® technology has now made it possible to encapsulate such biologically active macromolecules into biodegradable polymer microspheres while preserving biological activity (Chung, et al., Cancer Res., 74(19):5377-5385 (2014)).

Three different strategies to locally deliver biopharmaceutical drugs to the gut using oral administration of nanoparticles have recently been compared (Coco, et a., Int. J. Phar., 440:3-12 (2013) PLGA-PEG-mannose nanoparticles achieved highest accumulation in inflamed tissue. No such studies with PIN® encapsulated TGFβ have been performed, but our observations suggest that oral TGFβ/ATRA-loaded microspheres reduce gut inflammation through local induction of regulatory T-cells. Preservation of biological activity and improved stability are what makes PIN® a novel, unique and proprietary process and support continued development of the combination TGFβ/ATRA product as an oral immune-based therapy for IBD.

1 Spray Drying

Methods for forming microspheres/nanospheres using spray drying techniques are described in U.S. Pat. No. 6,620,617, to Mathiowitz et al. In this method, the polymer is dissolved in an organic solvent such as methylene chloride or in water. A known amount of one or more active agents to be incorporated in the particles is suspended (in the case of an insoluble active agent) or co-dissolved (in the case of a soluble active agent) in the polymer solution. The solution or dispersion is pumped through a micronizing nozzle driven by a flow of compressed gas, and the resulting aerosol is suspended in a heated cyclone of air, allowing the solvent to evaporate from the microdroplets, forming particles. Microspheres/nanospheres ranging between 0.1-10 microns can be obtained using this method.

2. Interfacial Polymerization

Interfacial polymerization can also be used to encapsulate one or more active agents. Using this method, a monomer and the active agent(s) are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

3. Phase Separation Microencapsulation

In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

a. Spontaneous Emulsion Microencapsulation

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

b. Solvent Evaporation Microencapsulation

Methods for forming microspheres using solvent evaporation techniques are described in E. Mathiowitz et al., J. Scanning Microscopy, 4:329 (1990); L. R. Beck et al., Fertil. Steril., 31:545 (1979); L. R. Beck et al Am J Obstet Gynecol 135(3) (1979); S. Benita et al., J. Pharm. Sci., 73:1721 (1984); and U.S. Pat. No. 3,960,757 to Morishita et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microspheres/nanospheres. This method is useful for relatively stable polymers like polyesters and polystyrene. However, labile polymers, such as polyanhydrides, may degrade during the fabrication process due to the presence of water. For these polymers, some of the following methods performed in completely anhydrous organic solvents are more useful.

4. Coacervation

Procedures for encapsulation using coacervation techniques are known in the art, for example, in GB-B-929 406; GB-B-929 40 1; and U.S. Pat. Nos. 3,266,987, 4,794,000, and 4,460,563. Coacervation involves the separation of a macromolecular solution into two immiscible liquid phases. One phase is a dense coacervate phase, which contains a high concentration of the polymer encapsulant (and optionally one or more active agents), while the second phase contains a low concentration of the polymer. Within the dense coacervate phase, the polymer encapsulant forms nanoscale or microscale droplets. Coacervation may be induced by different methods including a temperature change, addition of a non-solvent or addition of a micro-salt (simple coacervation), or by the addition of another polymer thereby forming an interpolymer complex (complex coacervation).

The amount of microgels needed to deliver a pharmaceutically effective dosage of insulin in a patient will vary based on such factors including but not limited to, the crosslinker and polymerizing group chosen, the protein loading capacity and efficiency of the gel particles, the toxicity levels of the biodegraded particles, the amount and type of bioactive material needed to effect the desired response, the subject's species, age, weight, and condition, the disease and its severity, the mode of administration, and the like. One skilled in the art would be able to determine the pharmaceutically effective dosage.

5. Phase Inversion Nano-Encapsulation (PIN®)

Phase Inversion Nano-encapsulation (PIN®) utilizes a non-mechanical microsphere encapsulation approach that preserves the structural integrity of macromolecules. PIN® allows the production of microspheres with an average size of 0.1-5 microns, ideally suited for oral delivery of agents to the gut, as particles smaller than 5 um in diameter readily traverse the GI barrier (Florence, et al., Pharm. Res., 14(3):259-266 (1997); Ermak, et al., Cell and Tissue Res., 279(2):433-436 (1995); Jani, et al., J. Pharm. Pharmacol., 41(12):809-812 (1989)). Orally administered PIN® microspheres are efficiently taken up in Peyer's patches and mesenteric lymph nodes (Chung, et al., Cancer Res., 74(19):5377-5385 (2014).

B. Methods of Using

The compositions disclosed herein are useful in treating subject inflammatory disorders of the gastrointestinal tract. Disorders which can be treated with the compositions disclosed therein include, but are not limited to Chrohns disease, ulcerative colitis, gastritis, collagenous colitis, lymphocytic colitis, diversion colitis, Behçet's disease/syndrome, indeterminate colitis, irritable bowel syndrome, and ileitis. The compositions disclosed herein can be used to prevent and/or ameliorate the inflammation-related complications associated with other autoimmune disorders such as type 1 diabetes.

The composition is administered to the subject following a therapeutically effective regimen, for length of time resulting in an improvement in one or more symptoms. For example, the composition can be administered one time per week, two times per week, or three times per week. In a preferred embodiment, the composition is administered three times per week. The subject is treated for a length of time effective to reduce one or more symptoms associated with inflammatory disorders of the gastrointestinal tract, for example, the severity of inflammation, extent of inflammation, disease associated fibrosis, disease associated weight loss, serum amyloid A levels etc. For example, the subject can be treated for 1 week, 2 weeks, 3 weeks, 4 weeks, 5 weeks, 6 weeks, 7 weeks, 8 weeks, 9 weeks or 10 weeks.

The combination therapy of TGFβ and ATRA when administered orally as disclosed herein, is more effective than either compound used alone and provides local delivery of the administered TGFβ and ATRA, with little or no systemic delivery of the administered dose, and is associated with reduced toxicity (as determined by the absence of therapy-associated fibrosis), when compared to systemic administration (which include higher doses of TGFβ and/or ATRA).

The major acute-phase protein serum amyloid A, SAA, is upregulated by a variety of inflammatory stimuli, including cytokines and glucocorticoids (GCs). Elevated systemic concentrations of SAA is a feature of inflammatory diseases, including inflammatory bowel disease. The compositions disclosed herein are effective to decrease SAA levels in treated subjects, when compared to untreated controls. Functionally, the compositions disclosed herein ameliorate the severity of symptoms associated with inflammatory conditions in the GI tract by increasing Foxp3 in CD⁺CD25⁺ in GI regulatory T-cells, when compared to untreated subjects.

The compositions can be administered alone or in combination with other bioactive agents. Exemplary bioactive agents include, but are not limited to sulfasalazine, -aminosalicylates (including mesalamine (Asacol, Lialda, Rowasa, Canasa, others), balsalazide (Colazal) and olsalazine (Dipentum)), corticosteroids such as prednisone and hydrocortisone; Azathioprine (Azasan, Imuran) and mercaptopurine (Purinethol, Purixan), Cyclosporine, Infliximab (Remicade), adalimumab (Humira) and golimumab (Simponi); Methotrexate, Natalizumab (Tysabri) and vedolizumab (Entyvio); Ustekinumab (Stelara); Metronidazole (Flagyl); Ciprofloxacin (Cipro); Anti-diarrheal medications, pain relievers, iron supplements, calcium, vitamin b-12 and vitamin D supplements. The present invention can be further understood in view of the following non-limiting examples.

### EXAMPLES

The studies below investigated whether oral administration of TGFβ and ATRA loaded PIN® microspheres could ameliorate disease in a murine model of IBD.

### Materials and Methods

### Preparation and Characterization of TGFβ1 and ATRA Loaded Microspheres.

Microsphere Preparation.

ATRA was encapsulated into polylactic-co-glycolic acid microspheres (1 mg of ATRA per gram of particles) using a modification of the solvent evaporation technique (Jeong, et al., Int. J. Pharma., 259(1-2):79-91 (2003)). Briefly, 5.0 mg of ATRA (Sigma-Aldrich) was dissolved in 20 ml DCM (dichloromethane) to produce a 250 mg/ml solution in an amber vial. 8 ml of this solution was added to 1000 mg of 503H polymer (Resomer) dissolved in 32 ml DCM to produce the oil phase. 20 ml of Span 80 was also pipetted into this solution. To prepare the water phase, 600 ml of 1% 25K PVA (polyvinyl alcohol) was added to a glass beaker placed on ice within a stainless steel container. The water phase was then set under a Silverson mixer at a shear rate of 4000 rpm for 75 minutes as the oil phase was added in droplets via a glass pipette. The emulsion was then transferred to the overhead mixer with an additional liter of double distilled water and spun for 225 minutes. The microspheres were washed, centrifuged, and lyophilized for 48 hours. TGFβ was encapsulated into poly-lactic acid (PLA) microspheres (0.285 mg TGFβ per gram of particles) using Phase-inversion (PIN®) technology as described previously (Egilmez, et al., Meth. in Mol. Med., 60(14):3832-3837 (2000)).

In Vitro Drug Release.

Human recombinant TGFβ (Peprotech, Rocky Hill, N.J.) was encapsulated into microspheres and release tested using an in vitro release assay described previously (Egilmez, et al., Cancer Res., 60(14):3832-3837 (2000). Briefly, 10 mg of TGFβ PIN® particles were suspended in 0.2 mL release buffer and transferred to the wells of a 96-well plate in triplicate. The plate was incubated at 37° C. in 5% CO2, the supernatant replaced daily and stored at −20° C. for use in TGFβ ELISA (Pierce-Endogen, Thermo-Fisher Scientific Inc, Rockford, Ill.) and bioassay (see below).

Stability Studies.

TGFβ particles were stored at 4 or -20° C., removed at selected time points and assayed for TGFβ bioactivity. For each time point, an overnight release assay was performed as above. TGFβ concentration and bioactivity values in supernatants were then compared with those observed for the same samples prior to storage (un-stored). Final values for specific activity were expressed as %-recovered bioactivity divided by %-recovered protein.

TGFβ biological activity was tested using the TGFβ sensitive mouse lymphoblast cell line HT-2. Cells were plated at 1.5×10⁴ cells/well in complete media containing 15 ng/mL m-IL-4. Standards and samples were added to the wells to produce a final volume of 200 μL/well. The 96 well plates were incubated at 37° C. and 5% CO2 for 67 hours. Cell numbers were determined using Promega Cell titer 96 Aqueous One Solution Reagent (Promega, Madison, Wis.) as per the manufacturer's instructions.

### SCID Mouse CD4+ T Cell Transfer Colitis Model

Animals:

Six to 8-week old BALB/c and CB-17 scid (SCID) mice were purchased from Taconic Farms (Germantown, N.Y.). The mice were kept under standard laboratory conditions with free access to food and water. They were allowed to adapt one week before starting the study. The care and use of laboratory animals was in accordance with a University at Buffalo IACUC-approved animal use protocol.

Isolation of CD4+ CD25− T-Cells:

CD4+ CD25− T-cells were purified from the spleens of naïve BALB/c mice by magnetic bead separation using MACS® column and separator according to manufacturer's instructions (Miltenyi Biotech, San Diego, Calif.). Purity and viability (>95%) were assessed by flow cytometry (FACScan, Becton Dickinson, San Jose, Calif.).

Induction of Colitis:

Purified CD4+ CD25− T-cells were adoptively-transferred to SCID recipients (4×10⁵ cells per mouse, IP). Mice were monitored twice daily for disease development and scored according to criteria in Table 1. Treatment started when mice reached an average score of 8-9 out of 25 (a score of 5 considered normal).

TABLE 1
Disease Scoring Criteria

| | | | Coat | | Fecal |
|---|---|---|---|---|---|
| Score | Dehydration | Activity | Condition | Posture | Consistency |
| 1 | Normal skin | Explores | Well | Normal | Normal stool; |
| | tent | environment | groomed | posture | small, |
| | | | coat | | firm and dry |
| 2 | Skin less elastic | Slight reduction | Slightly | Occasional | Small, firm, |
| | | in activity | ruffled coat | hunching | moist, |
| | | | | | adherent |
| 3 | Skin tent | Moderate | Moderately | Moderately | Larger, |
| | | reduction in | ruffled coat | hunched, | pliable, very |
| | | activity | | mobile | adherent stool |
| 4 | Skin tent + | No movement | Piloerected/ | Severely | Uniform liquid |
| | sunken eyes | without | poorly | hunched, | stool |
| | | stimulation | groomed | lethargic | |
| 5 | Persistent skin | Does not | Completely | Piloerection, | Liquid stool |
| | tent + SE | respond to | hunched, | no | with mucus |
| | | physical | peri-anal | movement | or blood |
| | | | stimulation | dermatitis | |

Isolation of Lymphocytes from Lamina Propria (LP)

Colonic LP lymphocyte populations were isolated by enzymatic digestion of minced colonic sections followed by purification on lympholyte as described (Berg, et al., J. Clin. Invest., 98(4):1010-1020 (1996). These CD4+ CD25+ T-cells were then analyzed for Foxp3-expression by flow cytometry (Gu, et al., Cancer Research, 70(1):129-138 (2010).

### Histology

Ascending, transverse and descending colon from treated and control mice were fixed in 10% formalin, embedded in paraffin, sectioned (5 μm) and stained with H & E. Five randomly-selected sections from each mouse were scored for disease severity using the scoring method outlined in Table 2.

TABLE 2
Histology Scoring Criteria
Severity of Inflammation

| | |
|---|---|
| 0 | None |
| 1 | Mild lymphoid infiltration |
| 2 | Lymphoid infiltration/focal crypt degeneration |
| 3 | Multifocal crypt degeneration and/or erosions |

Extent of Inflammation

| | |
|---|---|
| 0 | None |
| 1 | Mucosal |
| 2 | Submucosal |
| 3 | Transmural |

Amount of Mucus

| | |
|---|---|
| 0 | Normal |
| 1 | Slight decrease of mucus |
| 2 | Moderate decrease. Focal absence of mucus |
| 3 | Severe depletion of mucus |
| 4 | Total absence of mucus |

Degree of proliferation

| | |
|---|---|
| 0 | None |
| 1 | Mild increase in cell numbers and crypt length |
| 2 | Moderate increase. Focally marked increase |
| 3 | Marked increase: entire section |

### Serum Amyloid a Levels

Serum levels of amyloid A [SAA] were measured using an ELISA kit [BioSource, Inc. Camarillo, Calif.].

### Dextran Sodium Sulphate-Induced Colitis

Animals: Mice 6-8-weeks old (C57Black6 mice [males: n=6; females: n=6; 12 per group]) were purchased from Harlan Nossan, San Pietro al Natisone, Udine, Italy, and housed in a vivarium under pathogen-free conditions at the Department of Biomedical Sciences in the School of Medicine at the University of Catania [Catania, Italy]. The mice were kept under standard laboratory conditions with free access to food and water. They were allowed to adapt for 1 week before starting the study. The care and use of laboratory animals was in accordance with a University of Catania IACUC-approved animal use protocol.

### Induction of Colitis and Treatment

Colitis was induced with 3% dextran sodium sulphate [50 kDa; Sigma-Aldrich] in drinking water for 4 days. The first treatment was given 24 h before induction of colitis [Day −1] and continued every other day for 6 days.

### Disease Score, SAA Levels and Histology

Disease score, SAA levels and histological analysis were performed as above.

### Statistical Analysis

Significance (p<0.05) between experimental and control groups was determined using Student's t-test analysis. In experiments with multiple groups, homogeneity of inter-group variance was analyzed by ANOVA.

### Results

Release of TGFβ from PIN® microspheres over 4 days

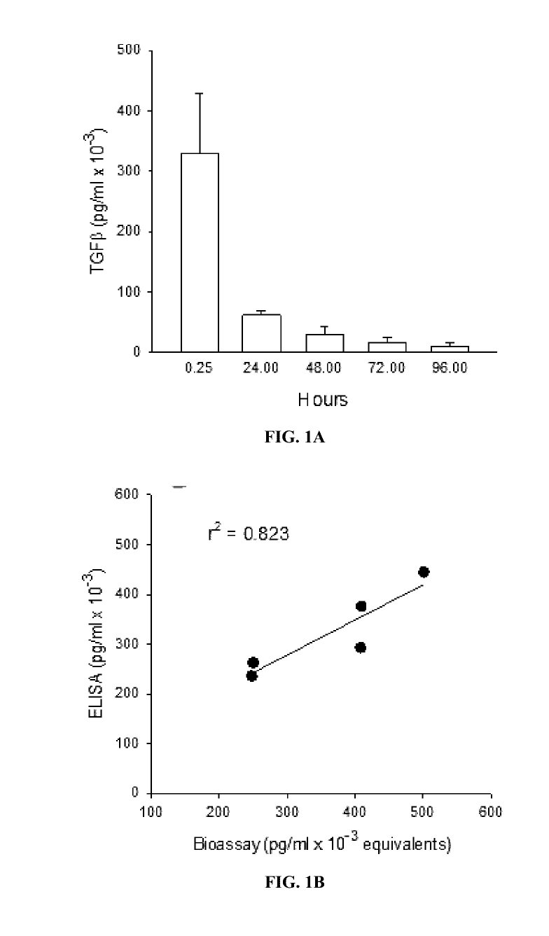

A number of technologies encapsulate small molecules such as ATRA achieving slow release and bioactivity (Jeong, et al., Int. J. Pharmaceutics, 259(1-2):79-91 (2003); Naahidi, et al., J. Control. Rel., 166(2):182-194 (2013)). The gentle encapsulation of labile proteins, protecting them from degradation, retaining bioactivity and shelf stability has been more elusive. Thus, the present studies focus on PIN® encapsulated TGFβ. The 4-day release profiles for human TGFβ from PIN® particles is shown in FIG. 1A. These data demonstrate that an initial burst was followed by sustained cytokine release for at least 4 days. To determine whether the released TGFβ was active, the 15-minute time point samples were evaluated for bioactivity (FIG. 1B). In this assay, the activity of the released protein was expressed as the concentration equivalent of unencapsulated TGFβ inducing that same amount of cell proliferation. Comparison of the cytokine concentrations as determined by bioassay versus ELISA revealed a high correlation (r²=0.823) between the two assays demonstrating that the specific activity of the encapsulated cytokine was fully preserved (FIG. 1B).

Bioactive TGFβ is Released from PIN® Microspheres after One-Year Storage at Either 4° C. or −20° C.

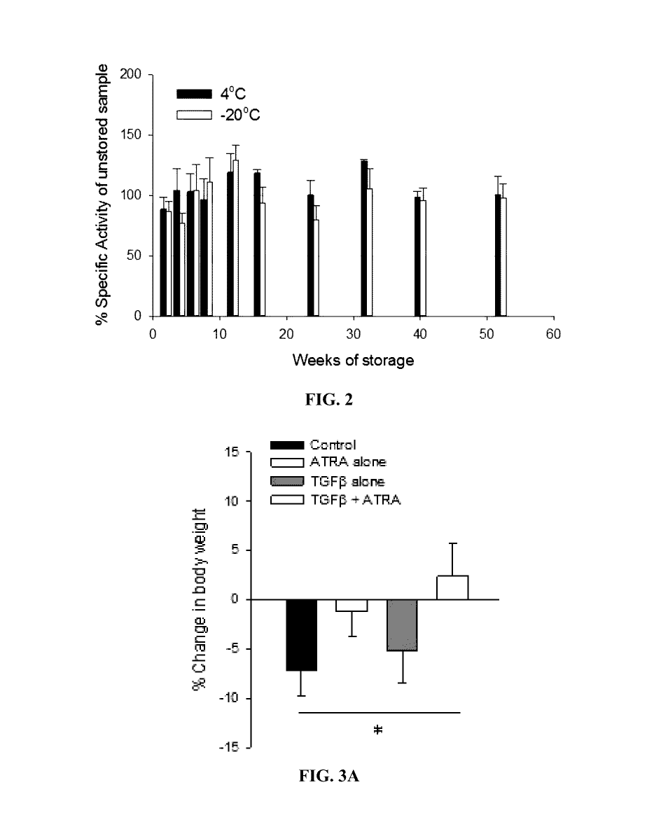

The bioactivity of the TGFβ released from PIN® microspheres stored for up to one year at 4° C. and −20° C. did not significantly differ from the bioactivity of the TGFβ released from fresh, unstored particles (FIG. 2). Overall these results show TGFβ loaded PIN® microspheres can be stored for at least one year at 4° C. or −20° C. with no significant reduction in the biological activity of encapsulated TGFβ.

TGFβ and ATRA Loaded PIN® Microspheres have Optimal Therapeutic Effects on IBD when Given in Combination

These studies first sought to determine if either ATRA-loaded microspheres alone or TGFβ loaded PIN® microspheres alone could ameliorate disease as effectively as the two in combination. Mice with established disease were weighed on day 0 and fed with blank microspheres (37.5 mg, control), ATRA (20 μg) loaded microspheres (20 mg) alone, TGFβ (5 μg) loaded microspheres (17.5 mg) alone, or with the combination. Treatment was 3 times a week for 2 weeks. Mice were weighed, sacrificed 4 days after last treatment for analysis of colons (colon samples prepared for histological analysis (five randomly selected sections from each mouse)) and sera.

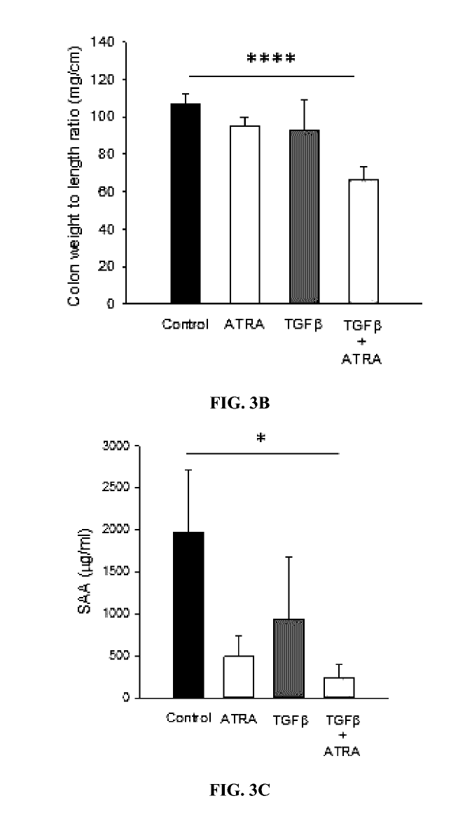

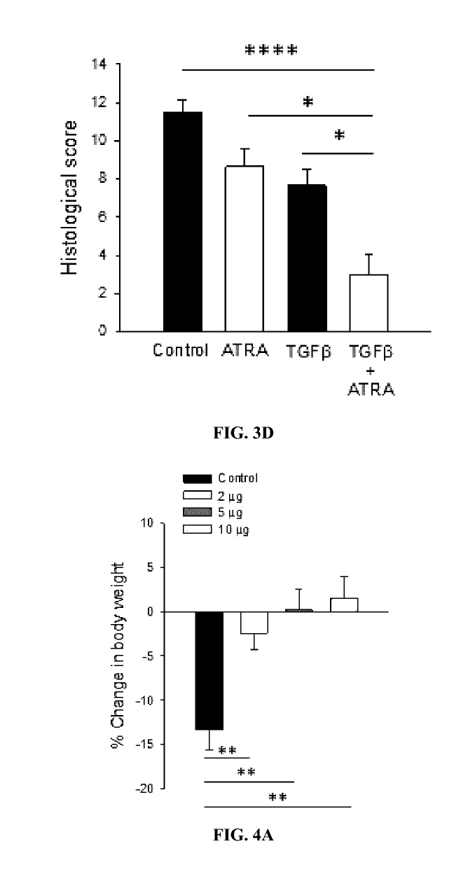

In contrast to single agent treatment groups, differences between control and ATRA/TGFβ treatment groups, in terms of percent body weight change (FIG. 3A), changes in colon weight to length ratio (FIG. 3B) and reduced SAA levels (FIG. 3C) were significant. In terms of histological scoring, the combination group performed significantly better than the control as well as either agent alone (FIG. 3D). Overall, combination therapy using ATRA and TGFβ produced more than additive results when all indicia measured where considered in combination.

### Effect of TGFβ or ATRA Dose on Therapeutic Efficacy

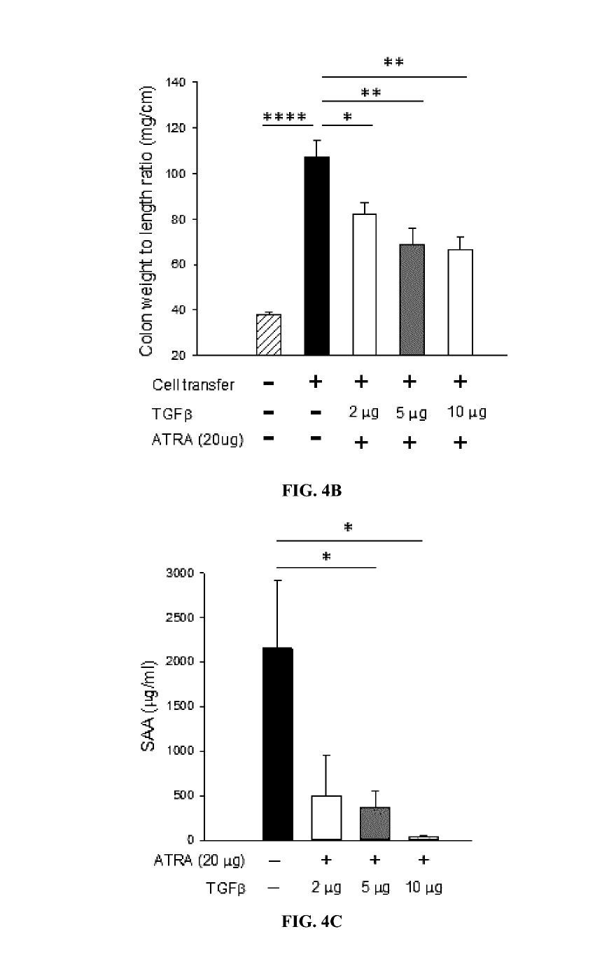

In the next set of experiments, using the same CD4+ CD25− T cell transfer model of IBD, dose response analysis was first performed for TGFβ. Mice with established disease were weighed (day 0) and treated orally with a constant dose of ATRA (20 g) loaded PIN® microspheres and increasing doses of TGFβ (2, 5 or 10 μg) loaded PIN® microspheres or blank microspheres (control) in 0.2 ml water 3 times per week. After 2 weeks, mice were weighed, sacrificed, serum taken and colons were weighed and measured. Selected disease markers were then monitored. The differences between control (blank microspheres) and treatment groups in terms of % body weight change (FIG. 4 A), colon weight to length (FIG. 4B) and SAA levels (FIG. 4C) were significant at all dose levels. However, by all three measures, the 10 μg TGFβ dose appeared most effective.

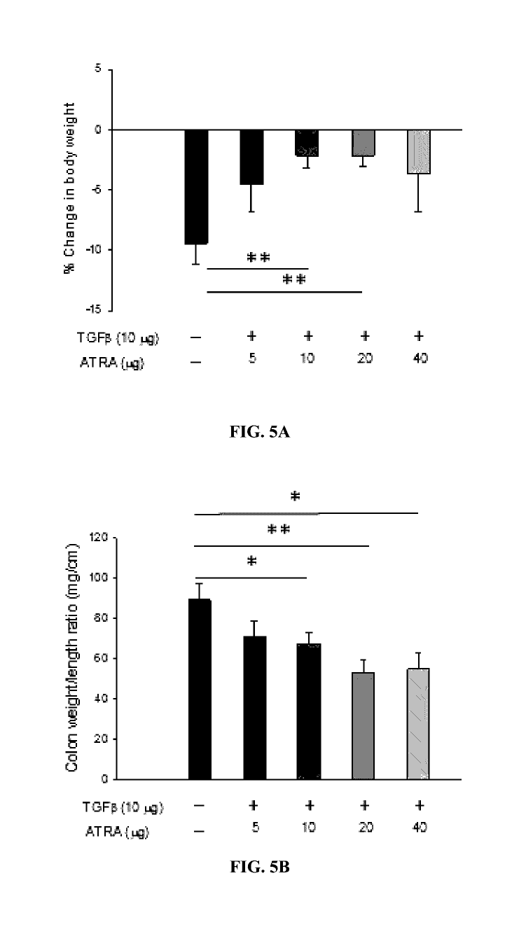

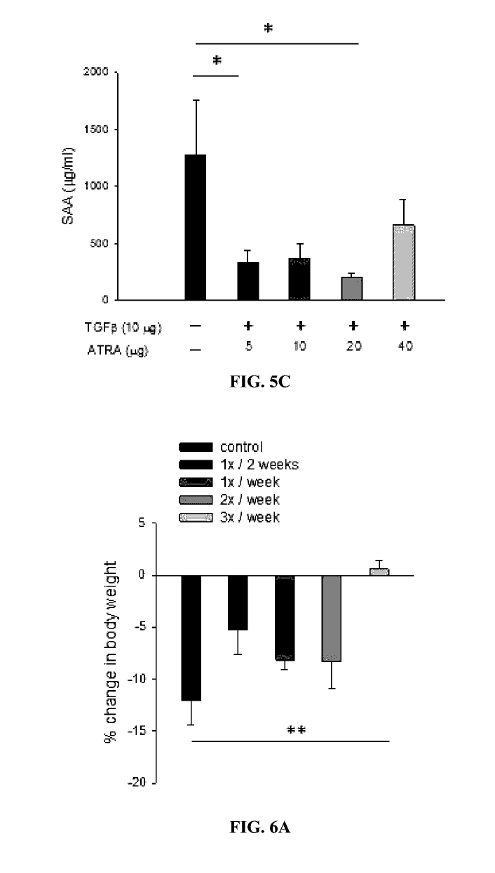

Next, keeping the TGFβ dose at 10 μg, a dose response analysis was performed for ATRA (FIGS. 5A-C). Mice with established disease were weighed (day 0) and fed TGFβ (10 μg) and ATRA (5-40 μg) microspheres, or blank microspheres (control) in 0.2 ml water 3 times per week. After 2 weeks, mice were weighed, sacrificed, serum taken and colons were weighed and measured. All doses of ATRA, except the highest dose tested, appeared to slow weight loss (FIG. 5 A) although the 10 and 20 μg doses seemed most effective. In terms of colon weight to length ratio (FIG. 5B), an apparent dose response was observed except the 40 μg dose, which was no more effective than the 20 μg dose. All doses of ATRA significantly reduced SAA levels (FIG. 5C), with the exception of the 40 μg dose. Differences between treatment groups were not generally significant. These observations show that at a TGFβ1 does of 10 μg, 20 μg ATRA is optimal.

### Effect of Dose Schedule on TGFβ/ATRA Therapeutic Activity

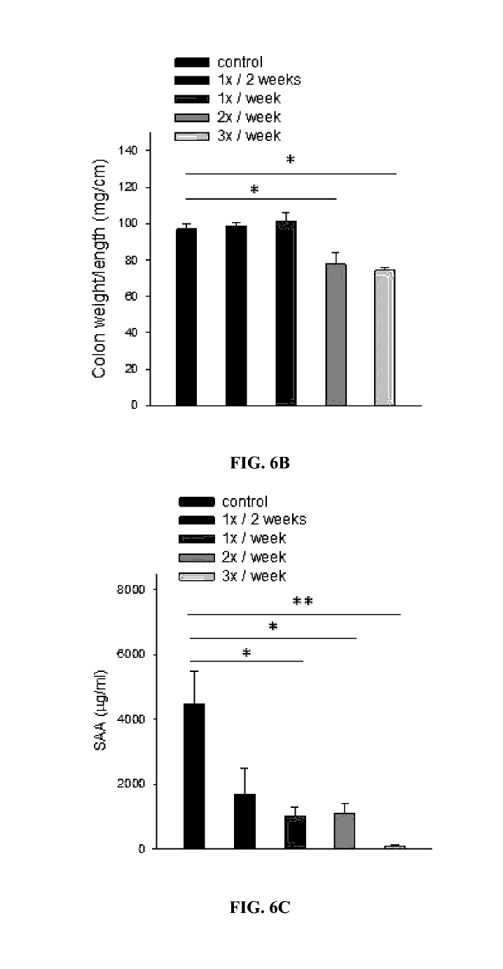

Having established that TGFβ/ATRA are effective at the doses discussed above, experiments were performed to determine an optimal treatment schedule. The same CD4+ CD25− T cell transfer method was used to induce IBD in SCID mice. When the mice became symptomatic for IBD, they were weighed, they were separated into groups as follows: Group 1: untreated controls; Group 2: mice that received oral TGFβ/ATRA 3 times per week (3×/wk); Group 3: treatment 2 times per week (2×/wk); Group 4: 1 treatment per week (1×/wk); and Group 5: one treatment over 2 weeks (1×/2 wk)). Each treatment consisted of the effective doses levels for TGFβ (10 μg) and ATRA (20 μg). After 2 weeks, mice were weighed, sacrificed, serum taken and colons were weighed and measured. In terms of maintenance of body weight (FIG. 6A), the 3×/wk dosing schedule was significantly different than control and performed best. A dosing schedule consisting of 3 treatments per week was also found to be superior by colon weight to length ratio (FIG. 6B) and reduced SAA levels (FIG. 6 C).

Treatment of Established IBD Disease with TGFβ/ATRA Loaded Microspheres

To more fully test the ability of oral TGFβ/ATRA loaded microspheres (10 μg and 20 μg, respectively) to ameliorate established IBD, a larger cohort study with frequent monitoring of five independent parameters was undertaken. Mice (n=6-9 per group) with established disease were weighed (day 0), administered TGFβ/ATRA loaded (10 μg and 20 μg, respectively) or blank microspheres (adoptive cell transfer [ACT] alone in 0.2 ml water; control) orally 3 times per week for 2 weeks and were monitored for overall disease score (Table 1) and weight loss 3 times a week. At the end of 2 weeks, animals were sacrificed 4 days after the last feeding, serum taken, colons weighed and measured; and colons samples prepared for histological analysis (five randomly selected sections from each mouse). Serum amyloid A (SAA) levels, colon weight-to-length ratios and colon histology scores determined.

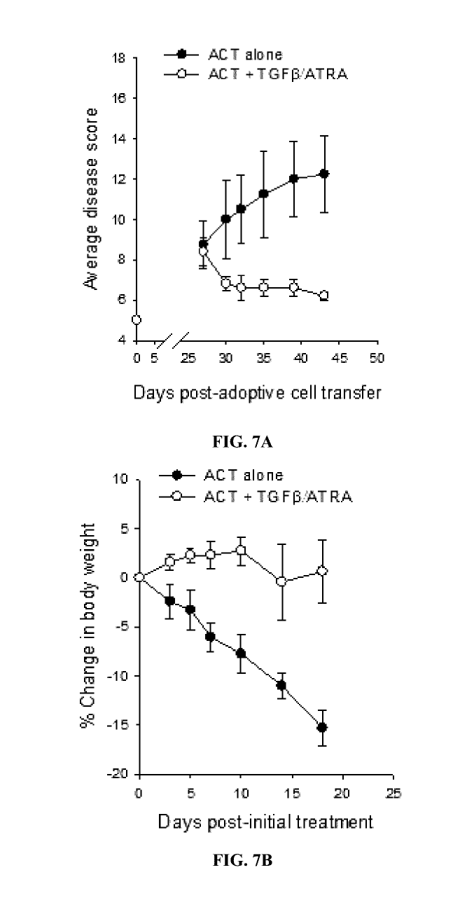

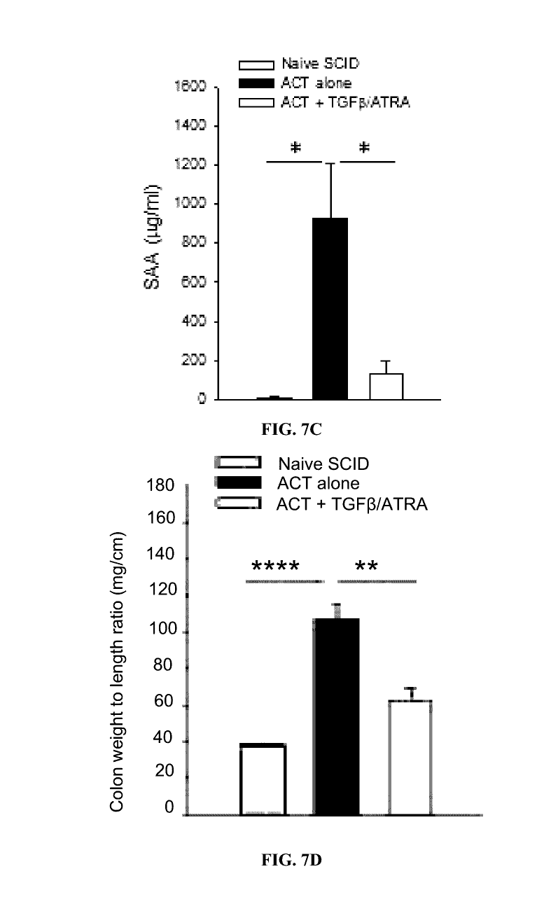

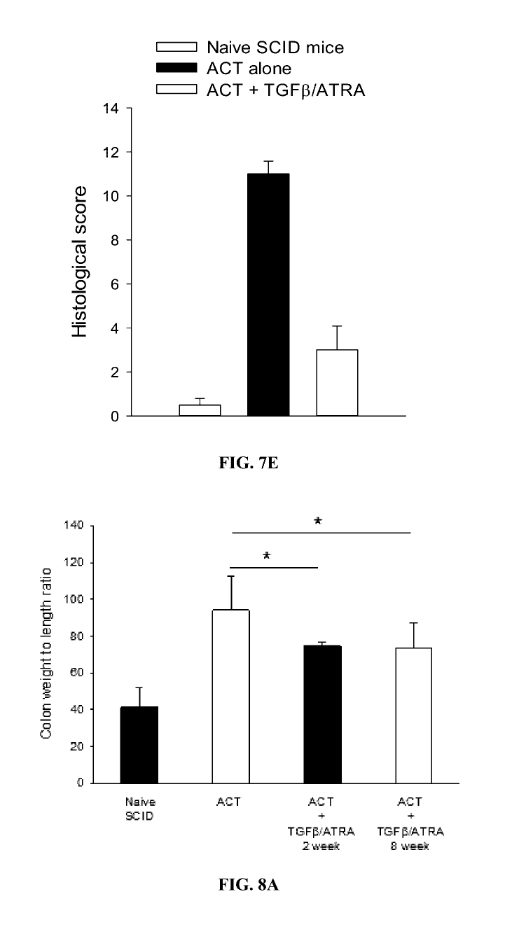

Treatment reversed disease rapidly as overall disease score declined within 4 days of treatment and remained low whereas it increased from an average of 8-9 to over 12 in control (ACT alone) mice during the 14-day treatment interval (FIG. 7A). Similarly, whereas the mice in the control group progressively lost weight (average of 17%) during this period, the weight remained stable in the treated group (FIG. 7B).

Mice were sacrificed on day 18 after first treatment (4 days after last treatment) and levels of SAA determined (FIG. 7 C). Age-matched naïve SCID mice (no adoptive cell transfer) were used to establish the baseline SAA levels (˜14 g/mL in naive SCID). These observations confirmed the above pattern of reduced disease score and suggested an even more dramatic reversal of disease. In control mice (ACT; fed blank particles alone) SAA levels increased over 50-fold to an average of ˜850 μg/mL. In contrast, TGFβ/ATRA treatment reduced SAA levels to −39 g/mL. Differences between naïve and control and naïve and TGFβ/ATRA treated groups were significant. Importantly, the difference between the control and the TGFβ/ATRA group was also significant (FIG. 7 C). The colons of sacrificed mice were also analyzed for colon length to weight ratio (FIG. 7 D). Colon weight to length ratio increased 2.7-fold in the control group in contrast to only a 1.6-fold increase in treated mice when compared to naïve SCID mice. Treatment resulted in a 45% decline in colon thickening.

Next, ascending, transverse and descending colon from naïve SCID TGFβ/ATRA-treated and control (fed blank particles) mice were fixed in 10% formalin, embedded in paraffin, sectioned (5 μm) and stained with H &E. Five randomly-selected sections from each mouse were scored (Table 2) for disease severity. A 22-fold increase in disease score was observed in control mice compared to naïve SCID mice, whereas the severity of disease was increased only by 6-fold in the treated group (FIG. 7E). The difference between naïve and control was highly significant. More importantly, the difference between control and TGFβ/ATRA treated mice was highly significant whereas naïve and TGFβ/ATRA-treated mice were not significantly different.

### Efficacy and Toxicity of Long-Term TGFβ/ATRA Microsphere Therapy

IBD was established in SCID mice using the same adoptive cell transfer model as before. Mice were considered to have IBD when they attained a disease score of 9 or above. At that time, mice were entered into control or treatment groups and treatments initiated.

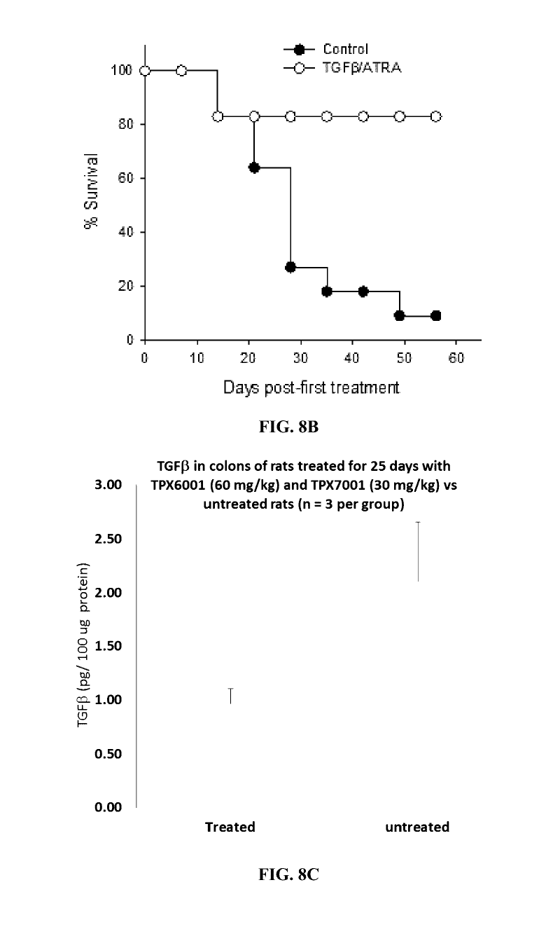

Mice with established disease [ACT] were treated either with control [ACT only] or TGFβ/ATRA particles for 2 weeks or 8 weeks and monitored for survival. All mice in the 2-week treatment group were euthanized at week 2. Treatments were administered for 8 weeks and consisted of three treatments each week of encapsulated TGFβ [10 μg] and ATRA [20 μg]. Mice that were in the control or 8-week treatment groups were euthanized upon reaching terminal disease criteria or at week 8. Mice were sacrificed if they developed rectal prolapse, their disease score exceeded a total of 17, or if they attained a score of 5 in any scoring category. The study was concluded and all remaining mice were sacrificed at 8 weeks [after 24 treatments]. Colons were harvested at euthanasia (between Weeks 48 in the control [ACT] group, at Week 2 in the 2-week treatment group and at Week 8 in the long-term treatment group) and analyzed for weight-to-length ratio. Values from age-matched naive controls are shown for comparison. The colon weight-to-length ratio of the control [ACT alone] mice was similar to that seen in control animals in earlier experiments regardless of the length of time with IBD [FIG. 8A]. Similarly, the mice receiving TGFβ/ATRA treatment for an extended 8-week period maintained the same colon weight-to-length ratio that is seen in mice that were treated for only 2 weeks. FIG. 8B, shows a clear survival advantage in mice receiving TGFβ/ATRA treatments with 83% of mice receiving TGFβ/ATRA therapy surviving to the study's end point of 8 weeks as compared with just 10% of mice treated with blank microspheres [control]. Lungs from mice receiving TGFβ/ATRA treatment for 8 weeks showed no statistically significant [p=0.84] difference in the lung-to-body weight ratio compared with untreated mice with IBD [data not shown], arguing against any fibrosis or systemic effect and for only therapeutic action. FIG. 8C shows the effect of oral treatment with TGFβNanoCap on levels of TGFβ in colon. Treated rats (n=3) received TGFβNanoCap (TPX6001 (TGFβ1) at 60 mg/kg and TPX7001 (ATRA) at 30 mg/kg) three times daily for four weeks. Small intestine, MLN (Mesenteric Lymph nodes), and colon of these animals were taken 4 hours after the final dose, along with tissues from age and sex matched untreated animals. All tissues were snap frozen at −20° C. and stored until used. Tissues were then thawed and homogenized using a glass tube with the pestle insert, in the presence of EDTA-free SIGMAFAST™ Protease Inhibitor Cocktail Tablets (used as per manufacturer's instructions). Levels of TGFβ in lysates were determined by ELISA kit according to manufacturer's instructions (Quantikine, R&D Systems, Minneapolis, Minn., USA). Data are expressed as ng/mL+/−Standard Deviation. *p=0.0065. TGFβ was not detected in either small intestine or MLN of either treated or untreated animals. However, the data indicate that TGFβNanoCap treatment reduced endogenous TGFβ levels in colon.

Increased Foxp3+ Expression in Colonic Lamina Propria CD4+CD25+T Cells from Treated Animals.

Mice in control and TGFβ1/ATRA treated groups were sacrificed following standard treatment and lamina propria CD4+ CD25+ T-cells were analyzed for Foxp3 expression by flow cytometry. CD4+ T cells were gated on and analyzed for CD25 and Foxp3 expression (dot plots). Histogram: Blue=CD4+ CD25− cells, Red=CD4+ CD25+ cells from control treated mice, Green=CD4+ CD25+ cells from TGFβ/ATRA treated mice. The data shows that oral treatment with TGFβ/ATRA particles induced a 45% increase (vs animals given blank particles) in Foxp3 MFI in CD4+ CD25+ colonic lamina propria regulatory T cells (FIG. 9). This is consistent with the current paradigm that TGFβ and retinoic acid synergize in inducing T-regulatory cell phenotype and function and provides support for the hypothesis that oral administration of sustained-release TGFβ and ATRA ameliorates established disease by shifting the CD4+ effector/Treg balance in favor of the regulatory phenotype in the GI tract.

### TGFβ/ATRA-Loaded Microspheres Attenuate DSS-Induced Colitis

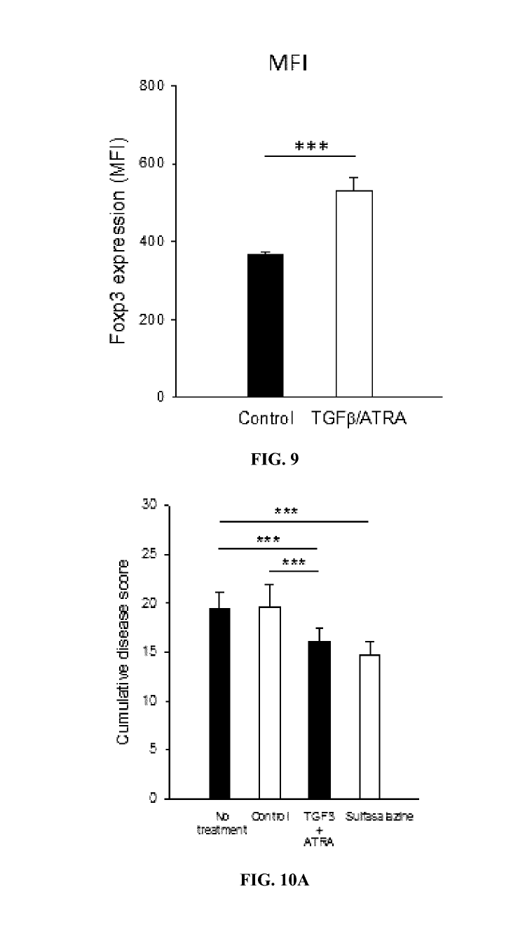

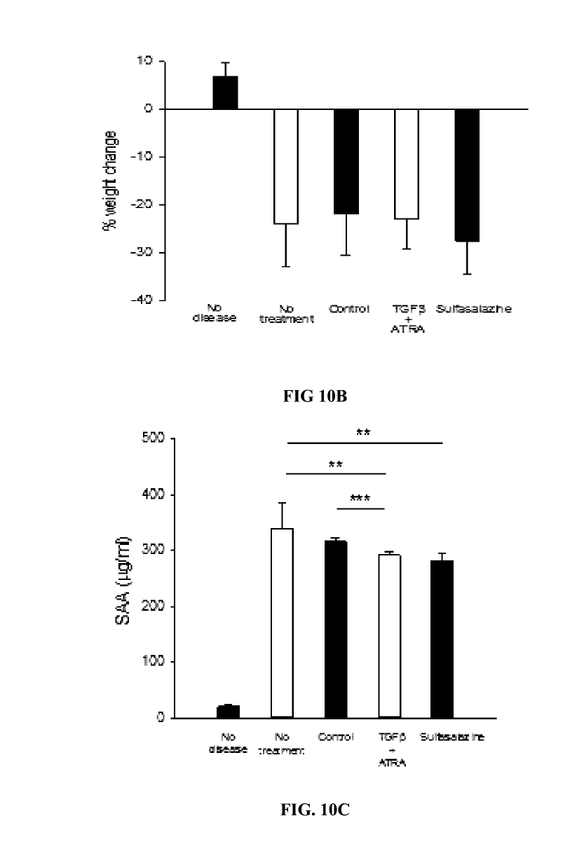

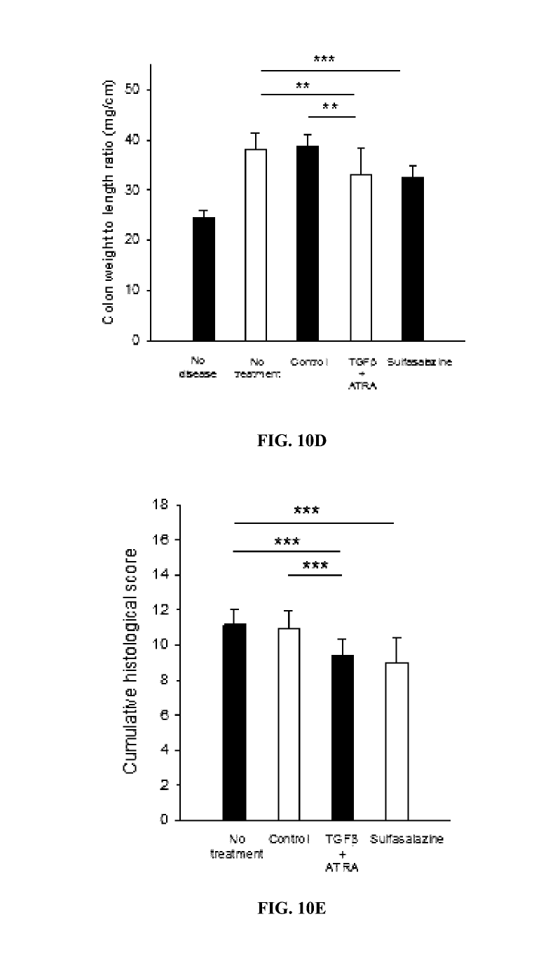

To more broadly test the ability of oral TGFβ/ATRA-loaded microspheres to ameliorate IBD, a second model, the DSS-induced colitis model, was employed. Acute colitis was induced in C57Black6 mice [equal numbers of males and females, 812 per group] with 3% DSS[50 kDa] in drinking water for 4 days. TGFβ/ATRA-loaded [10 and 20 μg, respectively] or blank microspheres [control] were fed [gavage] to mice [n=12 per group] 24 h before induction of colitis [Day −1] and then every other day for 6 days. Body weights were taken daily starting on Day 0. As a positive control group, eight additional mice were treated orally with sulphasalazine at the dose of 50 mg/kg daily for 7 consecutive days starting from Day 2. Mice were monitored daily for overall disease score and weight loss. At the end of 7 days, animals were sacrificed, sera taken and colons harvested, weighed, and measured on Day 7 [24 h after final treatment]. Colon samples were then prepared for histological analysis [five randomly selected sections from each mouse]. SAA levels, colon weight-to-length ratios, and histology scores determined. TGFβ/ATRA treatment attenuated disease as evidenced by significantly [p<0.001] reduced cumulative disease score compared with either untreated animals, or mice fed blank particles [control; FIG. 10A] however, neither sulphasalazine nor TGFβ/ATRA treatment was able to reduce weight loss in this model [FIG. 10B]. Mice were sacrificed 7 days after first treatment and levels of SAA determined [FIG. 10C]. Naive C57Black6 mice [no disease] were used to establish baseline SAA levels [˜20 μg/ml]. These observations confirmed the above pattern of reduced disease score. In control groups [mice left untreated or fed blank particles] SAA levels increased over 10-fold to an average of ˜350 μg/ml. In contrast, TGFβ/ATRA treatment reduced SAA levels to less than 300 μg/ml. Differences between no treatment and suphasalazine and TGFβ/ATRA-treated groups were significant. Importantly, the difference between the control and the TGFβ/ATRA group was also highly significant [p<0.001]. The colons of sacrificed mice were also analyzed for colon weight-to-length ratio [FIG. 10D]. Colon weight-to-length ratio increased to almost 40 mg/cm in the control groups in contrast to only 35 mg/cm in sulphasalazine- and TGFβ/ATRA-treated mice. This difference was also significant [p<0.01]. Next, ascending, transverse, and descending colon from naïve C57Black6 [no disease], untreated, TGFβ/ATRA-treated, and control [fed blank particles] mice were fixed in 10% formalin, embedded in paraffin, sectioned [5 μm] and stained with H & E. Five randomly-selected sections from each mouse were scored [Table 2] for disease severity. Cumulative histological score was nearly 12 in untreated and control mice, but was reduced to less than 10 in the treated groups [FIG. 10E]. The differences were highly significant [p<0.001]. Importantly, TGFβ/ATRA performed as well as the positive control drug sulphasalazine in this model.

In addition to the studies discussed above a bioavailability and pharmacokinetic study of TGFβ and ATRA was conducted following a single oral administration of PIN®-encapsulated TGFβ and ATRA in male rats, when compared to intravenous administration. Results argued against significant systemic TGFβ exposure after oral administration. After intravenous [IV] injection of soluble protein [0.04 mg/kg], serum levels of TGFβ peaked [35 000 pg/ml] at 5 min and declined rapidly over the first few minutes [t1/2=5 min]. Area under the curve [AUC] was 10 ng−hr/ml. These observations generally agree with the literature (Zioncheck, et al., Pharm Res, 11:21320 (1994). In contrast, for the oral TGFβ encapsulated group [100 mg/kg], very little TGFβ [˜500 μg/ml] was observed in serum and quickly declined to undetectable levels [Cmax˜30 min]. For ATRA, serum levels [˜3000 ng/ml] peaked at 5 min after IV injection of soluble drug [1.6 mg/kg] and declined rapidly over the first hour [t1/2=34 min]. AUC was 3.5 μg-hr/ml, consistent with the studies in Saadeddin, et al., AAPS PharmSciTech, 6:E1 (2004). The oral encapsulated ATRA group [18 mg/kg] also differed from the IV administration group: only a small amount [˜20 ng/ml] appeared in blood. Levels peaked at 60 min and rapidly declined with a t1/2 of 143 min [Auci et al., unpublished observations]. These findings are consistent with the conspicuous lack of fibrosis in the lungs of mice receiving long-term TGFβ/ATRA treatment. Nevertheless, risk remains that oral TGFβ could be responsible for the development of intestinal fibrosis during treatment for chronic colitis.

### DISCUSSION

The data shows that TGFβ loaded PIN® microspheres provide sustained release of bioactive protein in vitro for at least 4 days and that the formulation is stable at −204° C. and 4° C. for at least 52 weeks. The combination of TGFβ and ATRA diminished the symptoms of established IBD in both CD4+ CD25+ T− cell transfer and DSS-induced colitis models as evidenced by significant changes in multiple disease markers. Higher and more frequent doses were more effective and both were required to achieve maximal disease amelioration, which was associated with enhanced Foxp3 expression in lamina propria CD4+ CD25+ T-cells.

The systemic or local administration of TGFβ protects in several autoimmune disease models (Chernajovsky, et al., Gene Ther., 4(6):553-559 (1997); Kuruvilla, et al., PNAS U.S.A., 88(7):2918-2921 (1991); Moritani, et al., J. Clin. Invest., 102(3):499-506 (1998); Racke, et al., J. Immunol., 146(9):3012-3017 (1991); Prud'homme, et al., Lab. Invest., 87(11):1077-1091 (2007); Prud'homme, et al., J. Autoimmunity, 14(1):23-42 (2000)) but requires microgram doses.

Gene therapy approaches have economically produced effective amounts Chernajovsky, et al., Gene Ther., 4(6):553-559 (1997); Moritani, et al., J. Clin. Invest., 102(3):499-506 (1998)) but high systemic levels raise serious concerns including pulmonary fibrosis and scleroderma (Zhang, et al., Biol. Signals, 5(4):232-239 (1996); Anscher, et al., Lung Cancer, 19(2):109-120 (1998); Matrat, et al., J. Toxicol. and Environ. Health, Part A, 55(5):359-371 (1998); Zhang, et al., Transplant Int., 11(Suppl 1):S325-327 (1998); Haustein, et al., J. Eur. Acad. Dermatol and Venerol., 11(1):1-8 (1998)) chronic GVHD (Liem, et al., Transplantation, 67(1):59-65 (1999)) and glomerulonephropathies (Kitamura, et al., Nephrol Dial Transplant., 12(4):669-679 (1997)). Thus, fibrosis was an expected treatment side effect. However, surprisingly, the results suggested that any fibrosis was limited to that caused by disease at the time of treatment initiation. This is evidenced by the observation that long-term oral treatment with TGFβ/ATRA combination did not lead to an increased colon weight-to-length ratio, as would be expected in the case of treatment-induced fibrosis. Instead, results indicated that long-term treatment with TGFβ/ATRA slowed disease progression, prolonged survival, and did not increase intestinal fibrosis. Additionally, evidence that long-term oral treatment with TGFβ/ATRA did not induce lung fibrosis was the observation of no increase in lung-to-body weight ratio compared with untreated mice with IBD [data not shown].

ATRA or TGFβ loaded microspheres given alone had only modest effects that were not statistically significant in most cases. Both were required for broadly significant benefit, which was associated with enhanced Foxp3 expression by the colonic lamina propria CD4+ CD25+ T-cells. While not being bound by theory, this data suggests a requirement for ATRA in TGFβ-driven effects (Sun, et al., J. Exp Med., 204(8):1775-1785 (2007); Coombes, et al., Seminars in Immunol., 19(2):116-126 (2007); Benson, et al., J. Exp Med., 204(8): 1765-1774 (2007); Mucida, et al., Science, 317(5835):256-260 (2007)) and suggesting that ATRA is needed as a co-factor in TGFβ based IBD therapy. In the presence of ATRA and TGFβ, antigen activated naïve T cells become regulatory T cells (Coombes, et al., J. Exp. Med., 204(8):1757-1764 (2007); Benson, et al., J. Exp Med., 204(8): 1765-1774 (2007); von Boehner, et al., J. Exp Med., 204(8): 1737-1739 (2007) while TGFβ in the presence of IL-6, can promote Th-17 T cells (Mucida, et al., Science, 317(5835):256-260 (2007)). Mechanistically, ATRA increases FoxP3 expression by Treg cells and inhibits the development of Th17 cells by enhancing TGFβ-driven Smad3 signaling and inhibiting IL-6 and IL-23 receptor expression (Xiao, et al., J. Immunol., 181:2277-84 (2008)). This also suggests that another advantage to the combined product may be that ATRA tends to reduce the side effects of TGFβ.

Additionally, the data shows a reduction in endogenous levels of TGFβ in the colons of treated animals; an indication that the oral treatment delivers and ATRA signal that reduces pathological overproduction of TGFβ in the IBD gut, while maintaining sufficient levels of TGFβ in the gut to generate the regulatory T-cells that reduce gut inflammation.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A composition for reducing one or more symptoms associated with an inflammation-associated disorder of the gastrointestinal tract (GI), comprising an effective amount of transforming growth factor (TGF) β and all-trans retinoic acid (ATRA) in a pharmaceutically acceptable carrier for oral administration.

2. The composition of claim 1, comprising polymeric microparticles encapsulating the TGFβ and ATRA.

3. The composition of claim 2 wherein TGFβ and ATRA are encapsulated.

4. The composition of claim 3, wherein ATRA is encapsulated into poly(lactic-co-glycolic)(PLGA) acid microspheres, and TGFβ is encapsulated into poly(lactic acid) (PLA) microspheres.

5. The composition of claim 4 wherein TGFβ is encapsulated using phase inversion nano-encapsulation.

6. The composition of claim 1, the form of a tablet, capsule, solutions, suspension, or syrup.

7. The composition of claim 1, wherein TGFβ and ATRA are present in an effective amount to reduce one or more symptoms associated with an inflammation-associated disorder, selected from the group consisting of severity of inflammation, extent of inflammation, disease associated fibrosis, disease associated weight loss and serum amyloid A levels.

8. A method of treating a subject in need thereof, comprising administering the composition of claim 1 to the subject.

9. The method of claim 8 wherein the composition is administered once weekly, preferably, twice weekly, and more preferably, thrice weekly.

10. The method of claim 8, wherein the subject presents with an inflammation-associated disorder of the GI tract selected from the group consisting of Chrohns disease, ulcerative colitis, gastritis, collagenous colitis, lymphocytic colitis, diversion colitis, Behçet's disease/syndrome, Indeterminate colitis, irritable bowel syndrome, and ileitis.

11. The method of claim 10, wherein the composition is administered for a length of time effective to reduce one or more symptoms associated with the inflammation-associated GI tract disorder.

12. The method of claim 11, wherein the one or more symptoms are selected from the group consisting of severity of inflammation, extent of inflammation, disease associated fibrosis, disease associated weight loss and serum amyloid A levels.
